(19) United States
(12) Patent Application Publication
(10) Pub. No.: US 2024/0424088 A1
(43) Pub. Date: Dec. 26, 2024

(54) Nant COVID Vaccine Cross Reactivity

(71) Applicant: ImmunityBio, Inc., Culver City, CA (US)

(72) Inventor: Patrick Soon-Shiong, Culver City, CA (US)

(73) Assignee: ImmunityBio, Inc., Culver City, CA (US)

(21) Appl. No.: 18/882,219

(22) Filed: Sep. 11, 2024

Related U.S. Application Data
(63) Continuation of application No. 18/620,192, filed on Mar. 28, 2024.
Application No. 18/620,192 is a continuation of application No. 18/533,042, filed on Dec. 7, 2023, now Pat. No. 12,016,916.
Application No. 18/533,042 is a continuation of application No. 18/060,513, filed on Nov. 30, 2022, now Pat. No. 11,911,459.
(60) Provisional application No. 63/284,203, filed on Nov. 30, 2021.

(51) Int. Cl. A61K 39/215; A61K 39/00; A61P 31/14

(52) U.S. Cl. CPC A61K 39/215; A61P 31/14; A61K 2039/545; A61K 2039/572

(57) ABSTRACT

Recombinant SARS-CoV2 vaccine compositions and methods are presented that have unexpected cross-reactivity against a variety of other coronaviruses, and particularly against SARS-CoV1, MERS-CoV, OC43-CoV, and HKU1-CoV in addition to significant reactivity against SARS-CoV2A. Moreover, the vaccine compositions presented herein also produced cross-reactive memory B cells as well as cross-reactive memory T cells with cross-reactivity spanning a relatively wide range of different coronaviruses.

This application is a continuation of co-pending U.S. application Ser. No.: 18/620,192 filed Mar. 28, 2024, which is a continuation of U.S. Pat. No.: 12,016,916, filed Dec. 7, 2023, which is a continuation of U.S. Pat. No. 11,911,459, filed Nov. 30, 2022, which claims the benefit of the U.S. provisional application 63/284,203, filed Nov. 30, 2021. Each of these applications are incorporated by reference herein in its entirety.

### SEQUENCE LISTING XML

The content of the following file which was electronically submitted via EFS-Web along with the present application is incorporated by reference herein in its entirety: a computer readable form (CRF) of the Sequence Listing, file name: Seq_List_102538.0086US3.xml, created on Mar. 27, 2024, and having the size 61.8 KB.

### FIELD OF THE INVENTION

The field of the invention is vaccine composition and methods, especially as it relates to cross-reactive vaccine compositions that are effective for a variety of corona viruses.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

While SARS-CoV2 diagnostic tests have become available in relatively short time, numerous attempts to treat the disease have so far shown mixed or inconclusive results. Most typically, patients with severe symptoms are treated to maintain respiration/blood oxygenation. More recently, use of vaccination efforts and antibody cocktails (e.g., casirivimab and imdevimab) as well as newly developed antiviral agents such as paxlovid (Pfizer) or molnupiravir (Merck) have reduced the rate hospitalization and mortality. Nevertheless, the COVID19 mortality rate remained significant, particularly in elderly, immune compromised individuals, and individuals with heart disease, lung disease, or diabetes. Despite improvements in acute care, it has become apparent that containment of the disease is critically important as social distancing and other public health mitigation measures can provide only moderate relief. Such need for containment is particularly pressing as new virus mutants are bound to evolve over time, and it is anticipated that at least some of these mutants may escape currently known immune therapies.

Moreover, as can be seen from FIG. 1, protection of the recently introduced SARS-CoV2 RNA vaccine is not equally effective against variants of the SARS-CoV2 wildtype virus. In addition, as can be seen from FIG. 2, even where individuals were vaccinated early such as first responders and medical personnel, the protective effect against a new infection began to wane after a relatively short period of time.

In an effort to address this pressing need, numerous candidate anti-SARS-CoV2 vaccine compositions have been developed that target one or more proteins of the virus (see e.g., FIMMU 2020, 11:602256). For example, Sinovac and Sinopharm are currently testing inactivated virus vaccine preparations. Cansino Biologics, Janssen Pharma, Oxford University, and Garnaleya have developed vaccines based on a non-replicating adenoviral vector that encodes one or more viral proteins. Novamax produced a protein subunit-based vaccine. More recently, RNA-based vaccines from Moderna and Pfizer have been approved in several jurisdictions. Most of these vaccines induce at least some (typically non-sterile) immunity against infection leading to disease, but it is unclear whether protection is effective across different variants or even strains, whether protection is effective over several months, and/or if sufficient immune memory protects an inoculated individual over extended periods. In addition, it is unclear whether such vaccines generate clinically meaningful T cell-based responses. Unfortunately, and despite the relatively large number of vaccine formulations in development and use, none of the known vaccine compositions were shown to be cross-reactive against other coronaviruses such as MERS-CoV, OC43-CoV, or HKU1-CoV, thereby limiting the usefulness of such vaccines, and to elicit a durable memory B and T cell population.

Thus, even though various vaccine compositions and methods targeting coronaviruses are known in the art, all or almost all of them suffer from several drawbacks, particularly where the vaccine is highly specific against only a single variant of a specific strain. Therefore, there remains a need for improved coronavirus compositions and methods that are effective against a variety of coronavirus strains and variants thereof.

### SUMMARY OF THE INVENTION

The inventive subject matter is directed to various vaccine compositions and methods of generating an immune response against multiple coronaviruses, including SARS-CoV1, SARS-CoV2, MERS-CoV, OC43-CoV, and HKU1-CoV. Remarkably, the vaccine compositions presented herein targeting both S (spike protein) and N (nucleocapsid) of SARS-CoV2 exhibited unexpected cross-reactivity against a variety of other coronaviruses, and particularly against SARS-CoV1, MERS-CoV, OC43-CoV, and HKU1-CoV in addition to SARS-CoV2. Even more remarkably, the vaccine compositions presented herein also produced cross-reactive memory B cells as well as cross-reactive memory T cells with cross-reactivity spanning a relatively wide range of different coronaviruses.

In one aspect of the inventive subject matter, the inventor contemplates a method of eliciting in a subject a cross-reactive immune response against a coronavirus that includes a step of administering to the subject a recombinant vaccine composition in a prime and/or boost administration. In such method the recombinant vaccine composition has (a) a first portion encoding a severe acute respiratory syndrome (SARS) coronavirus nucleocapsid protein (N) that is fused to an endosomal targeting sequence (N-ETSD), wherein the first portion is functionally coupled to one or more regulatory elements that enable N-ETSD expression, and (b) a second portion encoding a SARS virus spike protein (S), wherein the second portion is functionally coupled to one or more regulatory elements that enable S expression. The vaccine composition is administered to the subject in an amount that elicits the cross-reactive immune response, wherein the cross-reactive immune response extends from SARS-CoV2 to a serologically distinct variant of SARS-CoV2, and/or to a coronavirus other than SARS-CoV2. Most typically, the coronavirus other than SARS-CoV2 is SARS-CoV1, MERS-CoV, OC43-CoV, and/or HKU1-CoV.

In some embodiments, the immune response is generation of antibodies that bind to at least two of the serologically distinct variants of SARS-CoV2 and/or to SARS-CoV2 and at least one coronavirus other than SARS-CoV2, and in other embodiments the immune response is generation of cytotoxic T cells that have cytotoxicity against different cells harboring respective serologically distinct variants of SARS-CoV2, and/or cells harboring SARS-CoV2 and cells harboring a coronavirus other than SARS-CoV2. In further embodiments, the immune response is generation of cross-reactive memory T cells, and in yet other embodiments the immune response is generation of cross-reactive memory B cells.

Preferably, the N protein is from SARS-CoV-2, and it is contemplated that the endosomal targeting sequence of the N-ETSD is encoded at a 5′-end of the first portion or at a 3′-end of the first portion. Moreover, it is preferred that the first and second portions are arranged in a bicistronic sequence. For example, the N-ETSD may have an amino acid sequence that has at least 90% identity to amino acid sequence SEQ ID NO:1 or have an amino acid sequence SEQ ID NO:1. In other examples, the first portion may have a nucleotide sequence SEQ ID NO:2.

With regard to the S protein it is contemplated that the S protein may have an amino acid sequence that has at least 90% identity to amino acid sequence SEQ ID NO:3 or SEQ ID NO:4, or that the S protein has amino acid sequence SEQ ID NO:3 or SEQ ID NO:4. For example, the second portion may have the nucleotide sequence SEQ ID NO:5 or the nucleotide sequence SEQ ID NO:6.

In further contemplated aspects, the recombinant vaccine composition may be formulated as a recombinant virus, and most preferably as an adenovirus having an E1 gene region deletion and an E2b gene region deletion. Alternatively, or additionally, the recombinant vaccine composition is formulated as a recombinant RNA, preferably a polycistronic RNA comprising the first and second portions. Where desired, the recombinant vaccine composition may also be formulated as a recombinant DNA that preferably comprises the first and second portions.

It is still further contemplated that the recombinant vaccine composition is administered in the prime and the boost administration. Preferably, but not necessarily, the recombinant vaccine composition is formulated as an adenoviral vaccine composition.

In yet other embodiments, the recombinant vaccine composition is administered only in the boost administration. In such case, the boost administration may follow a prime vaccination using a vaccine such as an RNA vaccine, a DNA vaccine, a viral vaccine, or a subunit vaccine. Exemplary RNA vaccine prime vaccination may be self-amplifying self-adjuvant RNA vaccines (that preferably comprise an RNA encoding a coronavirus S protein and/or a coronavirus N protein), and exemplary viral vaccine prime vaccination may comprise an adenoviral viral vaccine (that preferably comprises a recombinant nucleic acid encoding only a coronavirus S protein).

In another aspect of the inventive subject matter, the inventor contemplates a method of generating memory B cells and/or memory T cells having cross-reactivity against multiple distinct coronaviruses where the method includes a step of administering to a subject a recombinant vaccine composition in a prime and/or boost administration, wherein the recombinant vaccine composition has (a) a first portion encoding a severe acute respiratory syndrome (SARS) coronavirus nucleocapsid protein (N) that is fused to an endosomal targeting sequence (N-ETSD), wherein the first portion is functionally coupled to one or more regulatory elements that enable N-ETSD expression, and (b) a second portion encoding a SARS virus spike protein (S), wherein the second portion is functionally coupled to one or more regulatory elements that enable S expression. It is contemplated that the memory B cells produce antibodies that are cross reactive. Most typically, the vaccine composition is administered in an amount that elicits formation of the cross-reactive memory B cells and/or memory T cells. Most typically, the multiple distinct coronaviruses include SARS-CoV1, SARS-CoV2, MERS-CoV, OC43-CoV, and HKU1-CoV.

It is further generally preferred that the nucleocapsid protein N is from SARS-CoV-2, which may further include an endosomal targeting sequence at the 5′-end or the 3′-end. In further preferred aspects, the first and second portions are arranged in a bicistronic sequence. For example, the N-ETSD may have an amino acid sequence that has at least 90% identity to amino acid sequence SEQ ID NO:1, or have the amino acid sequence SEQ ID NO:1. Therefore, the first portion has nucleotide sequence SEQ ID NO:2.

The spike S protein preferably an amino acid sequence that has at least 90% identity to amino acid sequence SEQ ID NO:3 or SEQ ID NO:4, or has the amino acid sequence SEQ ID NO:3 or SEQ ID NO:4. Therefore, the second portion may have the nucleotide sequence SEQ ID NO:5 or SEQ ID NO:6.

As will be readily appreciated, the recombinant vaccine composition may be formulated as a recombinant virus (e.g., adenovirus having an E1 gene region deletion and an E2b gene region deletion) or may be formulated as a recombinant RNA (e.g., polycistronic RNA comprising the first and second portions), or may be formulated as a recombinant DNA (e.g., comprising the first and second portions).

Viewed from a different perspective, the inventor also contemplates a kit that includes a first recombinant vaccine composition that has (a) a first portion encoding a severe acute respiratory syndrome (SARS) coronavirus nucleocapsid protein (N) that is fused to an endosomal targeting sequence (N-ETSD), wherein the first portion is functionally coupled to one or more regulatory elements that enable N-ETSD expression, and (b) a second portion encoding a SARS virus spike protein (S), wherein the second portion is functionally coupled to one or more regulatory elements that enable S expression. The kit will also include a second recombinant vaccine composition that has (a) a recombinant viral vaccine comprising a recombinant nucleic acid encoding a SARS virus spike protein (S), functionally coupled to one or more regulatory elements that enable S expression; or (b) a self-amplifying self-adjuvant RNA vaccine comprising a recombinant nucleic acid encoding a SARS virus spike protein (S), functionally coupled to one or more regulatory elements that enable S expression, and optionally further encoding a severe acute respiratory syndrome (SARS) coronavirus nucleocapsid protein (N) functionally coupled to one or more regulatory elements that enable N expression; or (c) a subunit vaccine comprising a recombinant protein of a corona virus; or (d) a heat inactivated coronavirus vaccine composition.

Therefore, the inventors contemplate a recombinant vaccine composition for use as a vaccine that elicits in a subject a cross-reactive immune response against a coronavirus, characterized in that the recombinant vaccine composition has (a) a first portion encoding a severe acute respiratory syndrome (SARS) coronavirus nucleocapsid protein (N) that is fused to an endosomal targeting sequence (N-ETSD), wherein the first portion is functionally coupled to one or more regulatory elements that enable N-ETSD expression, and (b) a second portion encoding a SARS virus spike protein (S), wherein the second portion is functionally coupled to one or more regulatory elements that enable S expression. Preferably, the cross-reactive immune response extends from SARS-CoV2 to a serologically distinct variant of SARS-CoV2, and/or from SARS-CoV2 to a coronavirus other than SARS-CoV2.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic illustration depicting differences in efficacy of a SARS-CoV2 RNA vaccine against various strains of SARS-CoV2.

FIG. 2 is a schematic illustration depicting decline in protective effect of a SARS-CoV2 RNA vaccine.

FIG. 3 depicts a schematic of an exemplary recombinant hAd5 virus used for cross-reactive vaccine compositions and methods presented herein.

FIGS. 4A-4D depict exemplary results for antibody cross-reactivity in individuals after vaccination with the recombinant hAd5 virus of FIG. 3. FIG. 4A depicts cross-reactivity results for MERS-CoV, FIG. 4B depicts cross-reactivity results for HCoV-HKU1, FIG. 4C depicts cross-reactivity results for HCoV-OC43, and FIG. 4D depicts a time course for cross-reactivity.

FIG. 5 depicts exemplary results for memory B cells generated in non-human primates after vaccination with the recombinant hAd5 virus of FIG. 3 showing that hAd5 S+N induces cross reactive memory B Cells to N of SARS-CoV-2.

FIG. 6 depicts exemplary results for memory B cells generated in healthy human subjects after vaccination with the recombinant hAd5 virus of FIG. 3 showing that hAd5 S+N induces cross reactive memory B Cells to N of SARS-CoV-2.

FIG. 7 depicts exemplary results for memory T cells generated in healthy human subjects after vaccination with the recombinant hAd5 virus of FIG. 3 showing that hAd5 S+N induces cross reactive memory B Cells to N of SARS-CoV-2.

FIG. 8 depicts one exemplary prime-boost vaccine regimen using the recombinant hAd5 virus of FIG. 3.

FIG. 9 depicts an exemplary SASA vaccine composition suitable for use in a prime-boost vaccine regimen using the recombinant hAd5 virus of FIG. 3.

FIG. 10 depicts another exemplary prime-boost vaccine regimen using the recombinant hAd5 virus of FIG. 3.

FIG. 11 depicts an exemplary B and T cell cross reactivity for a universal COVID vaccine.

FIG. 12 depicts an exemplary validation of the need for S+N to induce long-term memory B & T cells for a universal 2nd generation vaccine.

FIG. 13 depicts an exemplary importance of N in generating T cell responses.

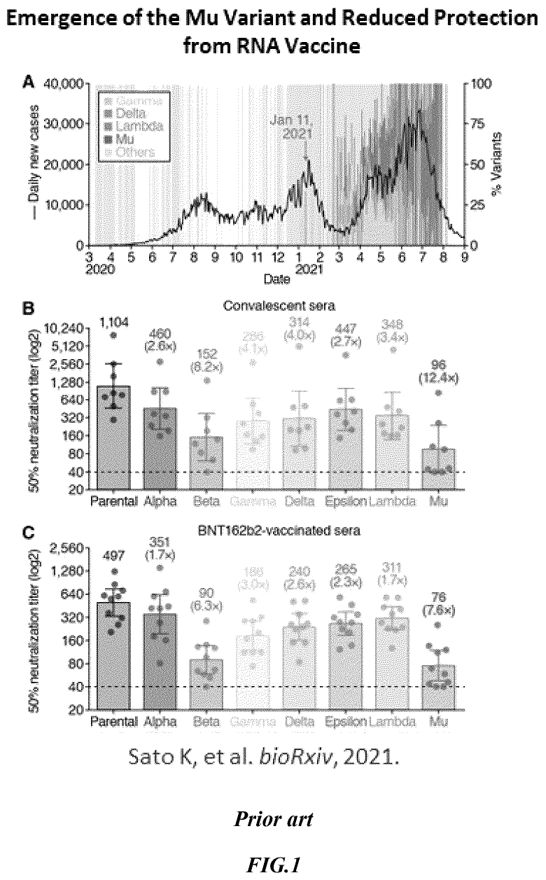

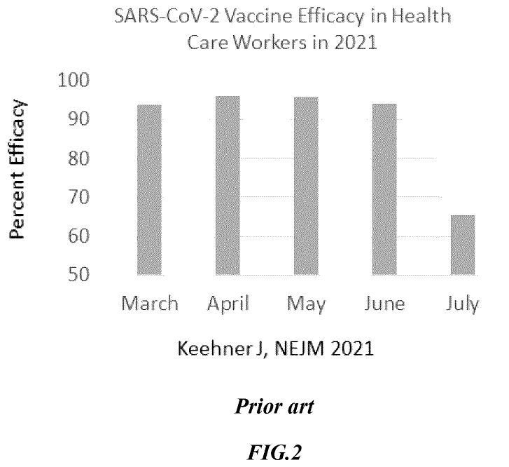

### DETAILED DESCRIPTION

The inventor has now discovered that various SARS-CoV2 vaccine compositions that included a nucleocapsid component unexpectedly elicited cross-reactive immune responses in human and non-human subjects upon administration, and particularly as boost administration. Notably, the cross-reactivity extended not only across different SARS-CoV2 strains but also to other members of the coronaviridae family, including SARS-CoV1, MERS-CoV, OC43-CoV, and/or HKU1-CoV. Even more notably, the cross reactivity was a durable response in which cross-reactive memory T cells and memory B cells were observed as is described in more detail below.

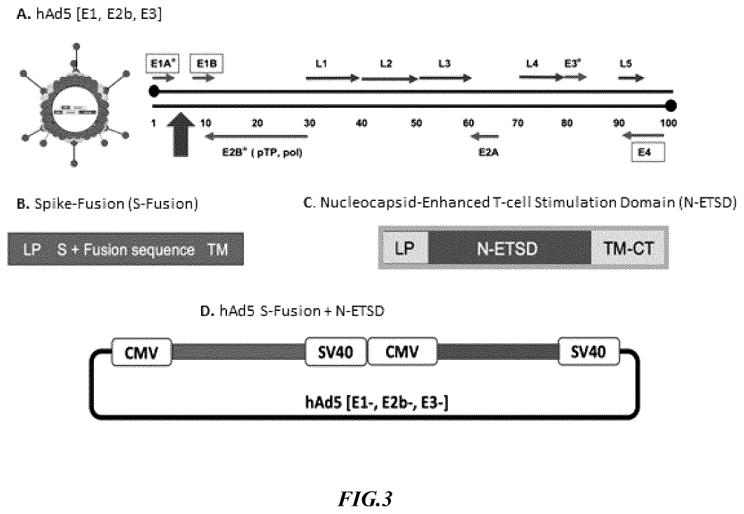

For example, one vaccine composition that included both a S component and an N component is shown in FIG. 3 in which the vaccine composition is formulated as a recombinant human adenovirus, and especially hAd5 with deletions in E1, E2b, and E3. Inserted into the viral genome is a recombinant nucleic acid that has a first segment that encodes an S-Fusion protein (comprising the S protein of SARS-CoV2 fused to a segment that enhances expression of the fusion protein) and a second segment that encodes N-ETSD (comprising the N protein of SARS-CoV2 and an endosomal targeting segment). As can be taken from FIG. 3, both S-Fusion and N-ETSD are under the control of a strong constitutive CMV promotor to so drive expression of the recombinant SARS-CoV2 proteins in a cell infected with the recombinant virus.

The above adenovirus-based vaccine comprising the hAd5 S-Fusion+N-ETSD used the unique and only clinically available human Adenovirus (hAd5) vector technology without adenoviral fiber production due to the deletions of the E1, E2b, E3 genes and allowed for a potent, long-lasting protein production for maximal cellular and humoral immunity. Moreover, such recombinant adenovirus had shown a proven safety profile in 13 Phase I/II clinical trials in over 125 elderly and immuno-compromised cancer patients. In addition, the recombinant adenovirus of FIG. 3 generated antigen specific CD4+ and CD8+ T cell in patients, even with previous adenoviral immunity. Thus, it should be appreciated that the recombinant adenovirus technology afforded a unique vaccine construct that maximized cell mediated immunogenicity and reduced the risk of antibody dependent enhancement. Still further, it should be recognized that such recombinant viruses can be prepared in high quantities using an established cell line, and that such vaccines are stable at simple refrigeration (2-8° C.).

While the recombinant viral vaccine construct is generally preferred in contemplated uses and methods, it should be recognized that numerous modifications can be performed lo long as the vaccine construct includes a N-protein component. Consequently, it should be appreciated that the recombinant constructs include recombinant viruses and recombinant yeasts, each of which contain a recombinant nucleic acid that will lead to expression of the N-protein (or modification and/or portion thereof) and S-protein (or modification and/or portion thereof).

In one embodiment, the N-ETSD polypeptide may comprises a sequence with at least 80% identity to SEQ ID NO:1. In other embodiments, the identity value is at least 85%. In still other embodiments, the identity value is at least 90%. In some embodiments, the identity value is at least 95%. In some embodiments, the identity value is at least 99%. In some embodiments, the identity value is 100%. It is further contemplated that the N-ETSD fusion protein contains a linker between the N-ETSD domain and the nucleocapsid protein. For example, this linker may be a 16 amino acid linker having the sequence (G₃S)₄. In certain embodiments, methods are disclosed herein for enhancing the immunogenicity of an intracellular antigen, the methods comprising tagging the antigen with ETSD and expressing the tagged antigen in an antigen-presenting cell (e.g., a dendritic cell).

In some embodiments, the fusion protein comprising N-ETSD and CoV-2 nucleocapsid protein may be encoded by a nucleic acid sequence having at least 80% identity to SEQ ID NO:2. In some embodiments, the identity value is at least 85%. In some embodiments, the identity value is at least 90%. In some embodiments, the identity value is at least 95%. In some embodiments, the identity value is at least 99%. In some embodiments, the identity value is 100%.

The CoV-2 spike protein is contemplated to have at least 85% identity to SEQ ID NO:3. In some embodiments, the identity value is at least 85%. In some embodiments, the identity value is at least 90%. In some embodiments, the identity value is at least 95%. In some embodiments, the identity value is at least 99%. In some embodiments, the identity value is 100%. The nucleic acid encoding the CoV-2 spike protein has at least 85% identity to SEQ ID NO:5. In some embodiments, the identity value is at least 85%. In some embodiments, the identity value is at least 90%. In some embodiments, the identity value is at least 95%. In some embodiments, the identity value is at least 99%. In some embodiments, the identity value is 100%.

The CoV-2 spike fusion protein is contemplated to have at least 85% identity to SEQ ID NO:4. In some embodiments, the identity value is at least 85%. In some embodiments, the identity value is at least 90%. In some embodiments, the identity value is at least 95%. In some embodiments, the identity value is at least 99%. In some embodiments, the identity value is 100%. The nucleic acid encoding the CoV-2 spike fusion protein has at least 85% identity to SEQ ID NO:6. In some embodiments, the identity value is at least 85%. In some embodiments, the identity value is at least 90%. In some embodiments, the identity value is at least 95%. In some embodiments, the identity value is at least 99%. In some embodiments, the identity value is 100%.

In a second aspect of this disclosure, provided herein is a recombinant yeast comprising a nucleic acid encoding a protein selected from the group consisting of a coronavirus 2 (CoV-2) nucleocapsid protein, a CoV2 N-ETSD protein, a CoV2 spike protein, a CoV2 spike-fusion protein, and a combination thereof. Moreover, each of these encoded proteins may be further modified as described in more detail below. Preferably, the recombinant yeast is Saccharomyces cerevisiae.

In some embodiments of this second aspect, the CoV-2 nucleocapsid protein or variant thereof comprises a sequence with at least 80% identity to SEQ ID NO:1 or SEQ ID NO:7. In other embodiments, the identity value is at least 85%. In still other embodiments, the identity value is at least 90%. In some embodiments, the identity value is at least 95%. In some embodiments, the identity value is at least 99%. In some embodiments, the identity value is 100%.

In some embodiment of this second aspect, the CoV-2 spike protein or spike fusion protein comprises a sequence with at least 80% identity to SEQ ID NO:3 or SEQ ID NO:4. In other embodiments, the identity value is at least 85%. In still other embodiments, the identity value is at least 90%. In some embodiments, the identity value is at least 95%. In some embodiments, the identity value is at least 99%. In some embodiments, the identity value is 100%.

In some embodiments, the nucleic acid encoding the CoV-2 spike protein or spike fusion protein comprises a sequence with at least 80% identity to SEQ ID NO:5 or SEQ ID NO:6. In other embodiments, the identity value is at least 85%. In still other embodiments, the identity value is at least 90%. In some embodiments, the identity value is at least 95%. In some embodiments, the identity value is at least 99%. In some embodiments, the identity value is 100%.

Most preferably, the recombinant virus is administered via subcutaneous or subdermal injection. However, in other contemplated aspects, administration may also be intravenous injection or intramuscular injection. In another aspect, the recombinant virus may be administered intranasally, for example via an intranasal spray. Alternatively, or additionally, antigen presenting cells may be isolated or grown from cells of the patient, infected in vitro, and then transfused to the patient.

In one aspect of any of the embodiments described above or elsewhere herein, the composition is formulated in a pharmaceutically acceptable excipient suitable for administration to a subject.

The immunotherapeutic compositions disclosed herein may be either “prophylactic” or “therapeutic”. When provided prophylactically, the compositions of the present disclosure are provided in advance of the development of, or the detection of the development of, a coronavirus disease, with the goal of preventing, inhibiting or delaying the development of the coronavirus disease; and/or generally preventing or inhibiting progression of the coronavirus disease in an individual. Therefore, prophylactic compositions can be administered to individuals that appear to be coronavirus disease free (healthy, or normal, individuals), or to individuals who has not yet been detected of coronavirus. Individuals who are at high risk for developing a coronavirus disease, may be treated prophylactically with a composition of the instant disclosure.

When provided therapeutically, the immunotherapy compositions are provided to an individual who is diagnosed with a coronavirus disease, with the goal of ameliorating or curing the coronavirus disease; increasing survival of the individual; preventing, inhibiting, reversing or delaying development of coronavirus disease in the individual.

In yet another embodiment, disclosed herein is a vaccine composition comprising the adenovirus or yeast as disclosed above, and wherein the composition is formulated for injection. The vaccine composition may be used for inducing immunity against CoV-2 in a patient in need thereof, by administering to the patient the vaccine composition.

Also disclosed herein are methods for preventing and/or treating coronavirus diseases, and especially COVID-19. Preferably, the method includes using a viral or yeast vector that encodes the wild-type or modified form of a nucleocapsid protein and/or the wild-type or modified form of a spike protein of the coronavirus in an immunogenic composition that is administered to a subject individual. The virus and/or yeast vaccine, thus administered, would infect the individual with CoV-2 the wild-type or modified form of the nucleocapsid or spike protein. With that in place, the individual would have an immune response against it, and be vaccinated. Notably, as the nucleocapsid protein and the spike protein are relatively conserved polypeptides, immune responses can be elicited for a variety of members of the coronavirus family.

Where the recombinant vector is an adenovirus, the adenoviral vector may be modified to encode the wild-type or modified form of the nucleocapsid protein, and/or spike protein. Similarly, in case of yeast, the yeast vector may also be modified to encode the wild-type or modified form of the nucleocapsid protein, and/or the spike protein. As is shown in more detail below, positive immune responses were obtained on cell mediated immunity upon administration of immunogenic compositions comprising the viral and/or yeast vectors in patients in need thereof. Thus, in one embodiment, the present disclosure contemplates creating the coronaviral spikes to be expressed on the yeast surface. In such embodiment, the yeast is acting as an avatar coronavirus to stimulate B cells, which then results in humoral immunity.

As disclosed herein is a next generation bivalent human adenovirus serotype 5 (hAd5) vaccine capable of inducing immunity in patients with pre-existing adenovirus immunity, comprising both an S sequence optimized for cell surface expression (S-Fusion) and a conserved nucleocapsid (N) antigen that is designed to be transported to the endosomal subcellular compartment, with the potential to generate durable immune protection. As further described herein, such bivalent vaccine has been found to be optimized for immunogenicity as evidenced by the following findings:
1) The optimized S-Fusion displayed improved S receptor binding domain (RBD) cell surface expression compared to S-WT where little surface expression was detected;
2) The expressed RBD from S-Fusion retained conformational integrity and recognition by ACE2-Fc;
3) The viral N protein modified with an enhanced T-cell stimulation domain (ETSD) localized to endosomal/lysosomal subcellular compartments for MHC I/II presentation; and
4) These optimizations to S and N (S-Fusion and N-ETSD) generated enhanced de novo antigen-specific B cell and CD4+ and CD8+ T-cell responses in antigen-naive pre-clinical models.

Both the T-cell and antibody immune responses to S and N components demonstrated a T-helper 1 (Th1) bias. The antibody responses were neutralizing as demonstrated by independent SARS-CoV-2 neutralization assays. Thus, in one embodiment, the next generation bivalent hAd5 S-Fusion+N-ETSD vaccine provides robust, durable cell-mediated and humoral immunity against SARS-CoV-2 infection. Moreover, and as also further described in more detail below, the vaccine construct may be administered orally, intranasally, or sublingually. Thus, in one embodiment, the instant disclosure also provides beyond injectable formulations (e.g., SC or IM) vaccine constructs in oral, intranasal, and sublingual formulation to induce mucosal immunity in addition to cell-mediated and humoral immunity. Viewed from another perspective, substantial immunity can be generated by injection, oral/mucosal administration, alone or in combination. In one embodiment, the COVID-19 vaccine disclosed herein generates long-term T and B cell memory. Further aspects, advantages and considerations suitable for use herein are disclosed in our copending International application publication with the publication number WO 2021/183665 (PCT/US21/21737), incorporated by reference herein in its entirety.

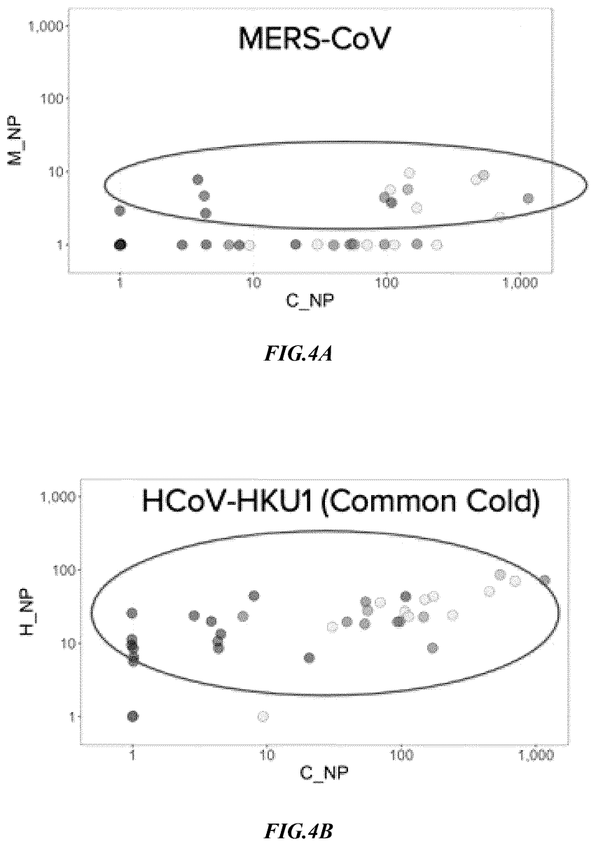

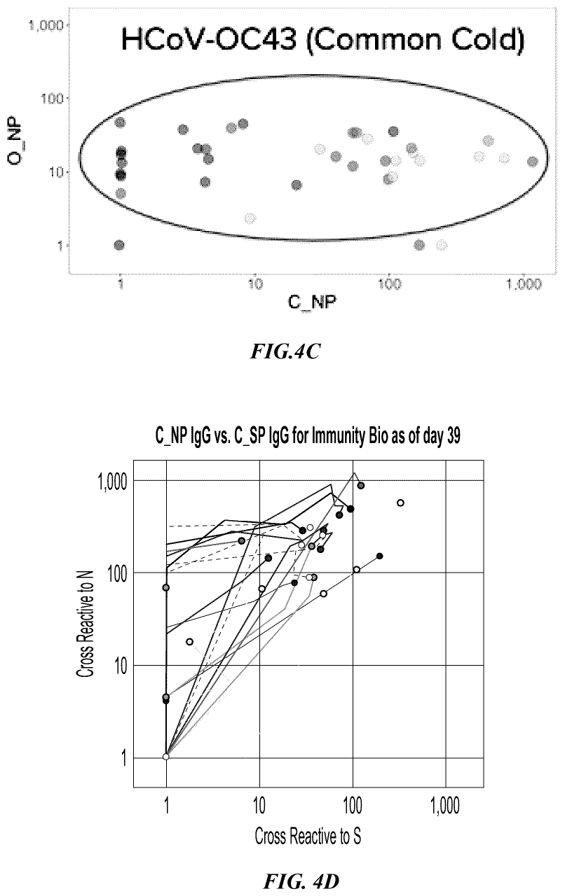

Using the above adenoviral hAd5 S+N vaccine composition as schematically shown in FIG. 3 in a prime and boost regimen in human (healthy volunteers), the inventor discovered that the vaccine composition after boost elicited not only a robust immune response against S and N of SARS-CoV2, but that the antibodies of the vaccinated human also had significant cross-reactivity against other coronaviruses, and especially against MERS-CoV, HcoV-HKU1, and HCoV-OC43 as is exemplarily shown in FIG. 4A, FIG. 4B, and FIG. 4C, respectively. When observing the time course of antibody generation in the vaccinated volunteers, it was observed that the anti-N antibodies rapidly increased relative to anti-S antibodies as can be seen in FIG. 4D. Such finding was entirely unexpected, is attributed to the presence of N as a component in the vaccine, and possibly also attributable to the ETSD sequence that was coupled to the N-protein, directing the N protein to the endosomal presentation pathway via MHC-II and thereby triggering a robust CD4+ response.

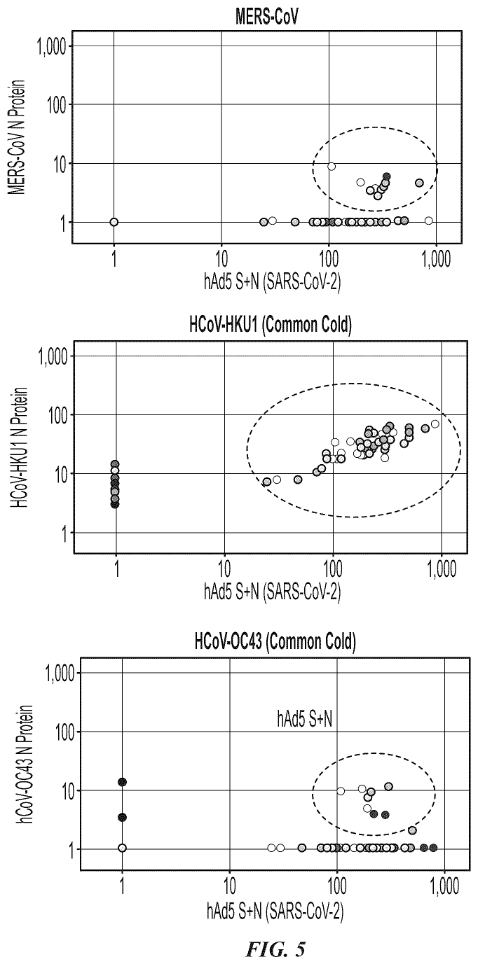

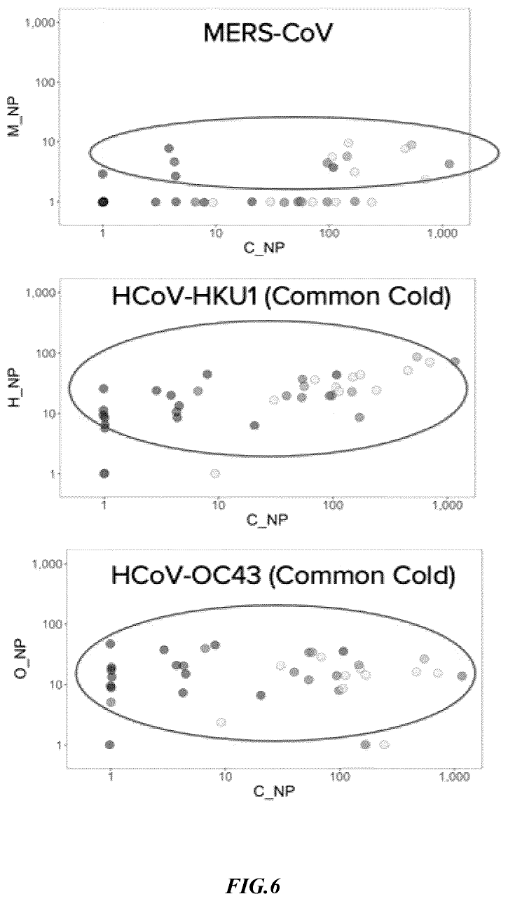

Following up on these results, the inventor then sought to identify whether or not the vaccine compositions presented herein would also elicit cross-reactive memory B cells to N in response to the vaccination. Remarkably, the hAd5 S+N vaccine once more elicited generation of cross-reactive memory B cells as is show in the exemplary data of FIG. 5. Here, cross-reactivity was observed against MERS-CoV, HcoV-HKU1, and HCoV-OC43. Similarly, where healthy human volunteers were subjected to prime and boost vaccination with the hAd5 S+N vaccine, the vaccine induced formation of memory B cells as is shown in the exemplary data of FIG. 6. Here once more, cross-reactivity was observed against MERS-CoV, HcoV-HKU1, and HCoV-OC43.

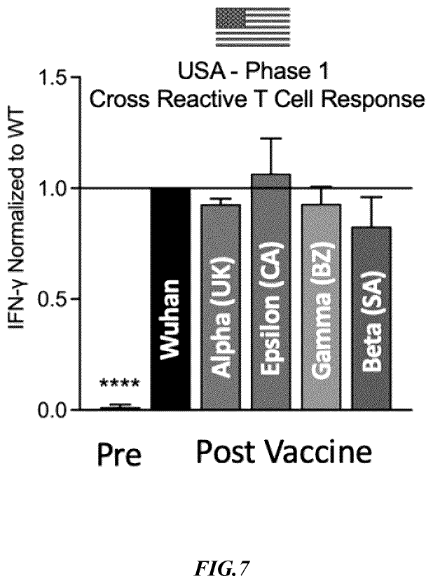

A further set of experiments was then conducted to determine whether the hAd5 S+N vaccine would induce formation of cross-reactive memory T cells in healthy human volunteers, and exemplary results are shown in FIG. 7. As is readily apparent, the vaccine was effective not only against the wildtype variant, but also across a wide spectrum of variants.

While the above experimental data were obtained under protocols that used the hAd5 S+N vaccine in both prime and boost administrations, it should be appreciated that the vaccine formulations presented herein are suitable for either prime or boost. However, it is especially contemplated that the vaccine compositions presented herein are particularly beneficial where they are employed in a boost administration following a prime administration that may or may not include an N-component. Therefore, contemplated prime vaccine administrations that can be followed with the vaccine composition presented herein include those targeting the S-protein, a fragment of the S-protein (and especially fragments comprising the RBD of the S protein), and/or fusion proteins of the S-protein or fragment thereof.

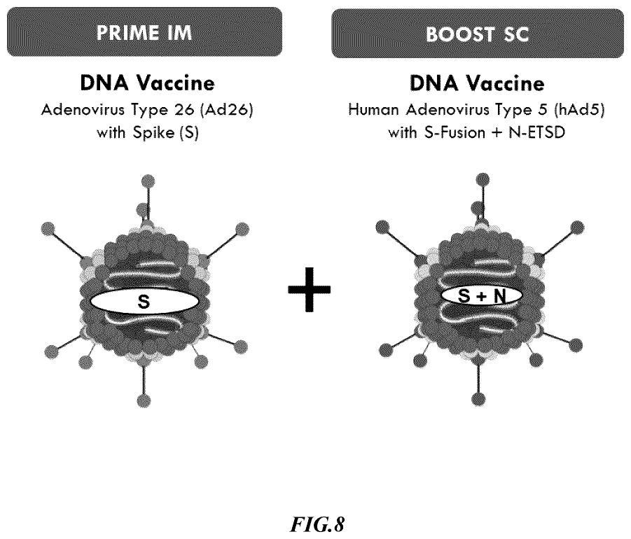

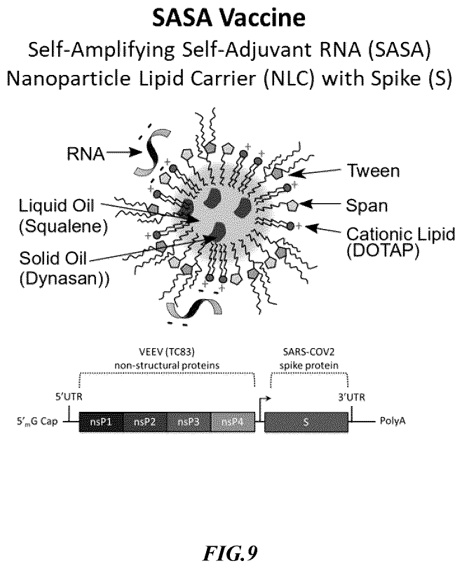

For example, a suitable prime/boost regimen is schematically depicted in FIG. 8 where the prime vaccination uses a recombinant adenovirus (here: Ad26) that includes a nucleic acid encoding the S protein. The boost vaccination the uses the hAd5 S+N vaccine as schematically shown in FIG. 3. Alternatively, the prime vaccination need not be based on a recombinant virus as described above but may also employ a SASA-type vaccine composition in which a nucleic acid encoding the S and/or N protein is coupled to a lipid carrier to so form a self-amplifying self-adjuvant RNA or DNA vaccine as exemplarily shown in FIG. 9. SASA-type vaccines have a variety of benefits over nanoparticle-based RNA vaccines (e.g., such as those provided by Pfizer or Moderna). The table below illustrates exemplary benefits for SASA-type vaccines in contrast to nanoparticle-based RNA vaccines.

| Limitation | Current RNA Vaccines | ImmunityBio RNA Vaccines |
|---|---|---|
| Storage/ | Requirement for deep- | NLC formulation allows for |
| Distribution | cold chain. | storage at room temperature |
| | | for years |
| Potency | Elicit immunity at levels | Self replicating RNA allows |
| | similar to recovered | for increased potency, |
| | patients, which may | allowing for potential single |
| | allow re-infection. | shot protection |
| Duration of | Modest immunogenicity | Self-Adjuvanting RNA |
| Immunity | may be associated with | vaccine platform may |
| | short durability | increase duration and |
| | | breadth of immunity |
| Protection | RNA sequence | RNA decorated on outside |
| against | encapsulated within | of NLC, allowing for easy |
| mutant | delivery vehicle making | swapping of genetic |
| SARS-CoV-2 | adaptations to new | sequence. |
| strains | strains challenging | Demonstrated ability to |
| | | vaccinate with multivalent |
| | | strains |

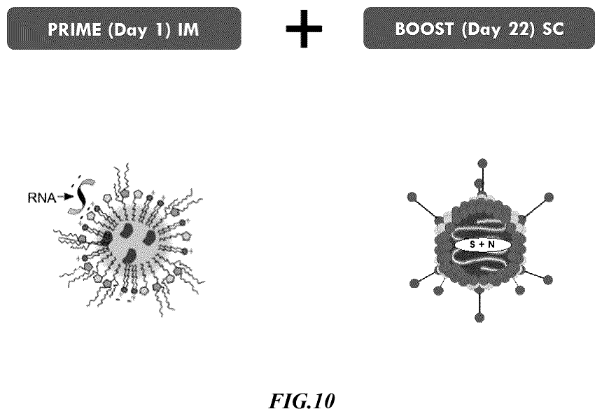

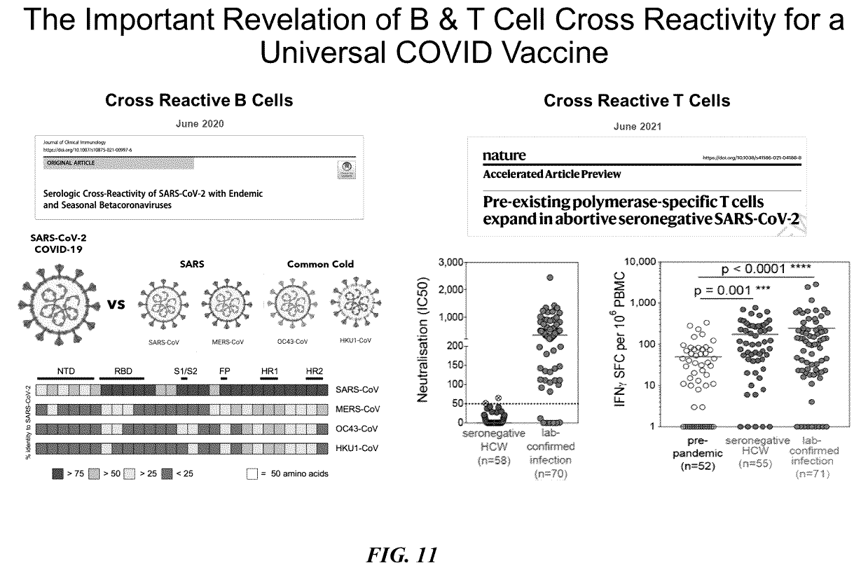

Therefore, the inventor also contemplates use of a SASA-prime vaccination as exemplarily shown in FIG. 10, followed by a recombinant viral boost vaccination using the hAd5 S+N vaccine as exemplarily shown in FIG. 3. In this context, it should be appreciated that a heterologous prime boost (“Mix and Match”) vaccine regimen has been shown to elicit some of the strongest and potentially most durable immune responses to COVID. In particular, a “Prime” vaccine with an RNA vaccine led to strong antibody response, while a “Boost” vaccine with a recombinant adenovirus vaccine makes for strong cellular immune responses. Such vaccine strategy as exemplarily outlined in FIG. 10 is believed to deliver a strong antibody response: Potent Th1 antibodies to both wildtype and beta variant, and a strong immune cell response: Potent CD8+ T cells to both S and N for wildtype and beta variant, and potent CD4+ T cells to both S and N for wildtype and beta variant.

Therefore, it is contemplated that any given prime vaccination against SARS-CoV2 can be substantially augmented with a boost vaccination using the hAd5 S+N vaccine as exemplarily shown in FIG. 3 (or other vaccine formulation that includes an N-component). Indeed, the hAd5 S+N vaccine is also deemed to be suitable where an individual has already received a prime and boost vaccination (e.g., a Pfizer, Moderna, or Johnson & Johnson vaccine). Such additional boost is believed to confer the same advantages with regard to cross-reactivity and memory B and memory T cell formation.

In still further contemplated aspects of the inventive subject matter, and particularly where the recombinant S and/or N protein is expressed in yeast or another suitable expression systems, the recombinant protein(s) can be combined as subunit vaccines with adjuvant 3M-052-Alum (which was developed by IDRI and 3M). As was unexpectedly observed, the 3M-052-Alum adjuvant also elicited significant cross-reactivity against other SARS-CoV variants and even other coronaviruses. Therefore, the N/N-ETSD and S/S-Fusion sequences presented herein are particularly contemplated for such subunit vaccines having the 3M-052-Alum adjuvant.

The important revelation of B and T cell cross reactivity for a universal COVID vaccine is illustrated in FIG. 11. Hicks J, et al (Serologic cross-reactivity of SARS-CoV-2 with endemic and seasonal Betacoronaviruses. J Clin Immunol. 2021 Mar. 16, which is incorporated by reference herein) discloses the cross-reactivity potential of SARS-CoV-2 antibodies with the full spike proteins of four other Betacoronaviruses that cause disease in humans, MERS-CoV, SARS-CoV, HCoV-OC43, and HCoV-HKU1. It was found that there was potential cross-reactivity of antibodies against SARS-CoV-2 towards the four other coronaviruses, with the strongest cross-recognition between SARS-CoV-2 and SARS/MERS-CoV antibodies, as expected based on sequence homology of their respective spike proteins.

The results disclosed herein support the inclusion of non-spike antigens in second-generation vaccines. In particular, the T cells induced by common cold coronaviruses play a protective role against SARS-COV2 infection. These T cells provide protection by attacking proteins within the virus, rather than the spike protein on its surface. The spike protein is under intense immune pressure from vaccine-induced antibody which drives evolution of vaccine escape mutants. In contrast the internal proteins targeted by the T cells mutate much less. Consequently, they are highly conserved between the various SARS-CoV-2 variants, including Omicron. Thus, the presently disclosed vaccines, which induce broadly protective T cell responses, provide a better protection against current and future SARS-CoV-2 variants.

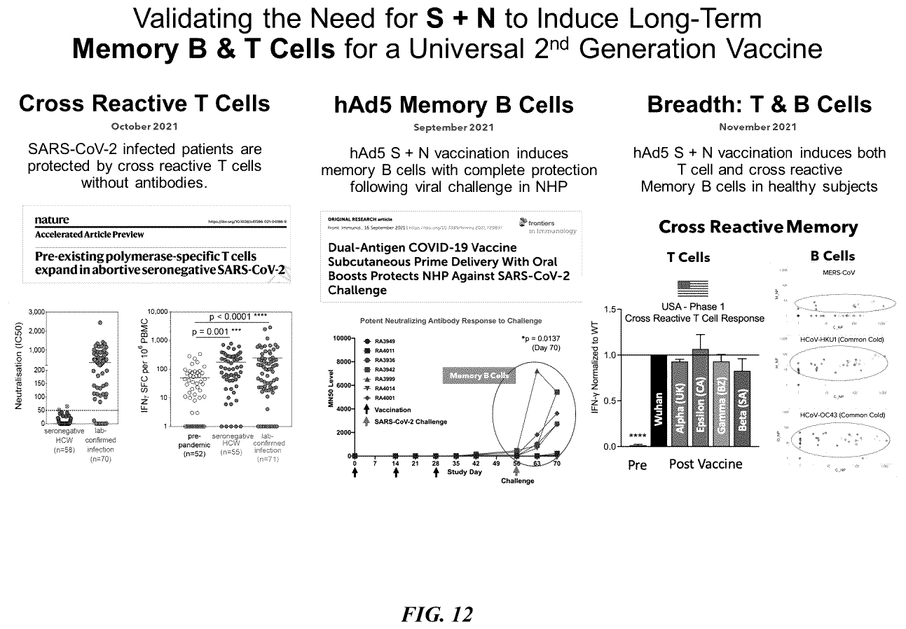

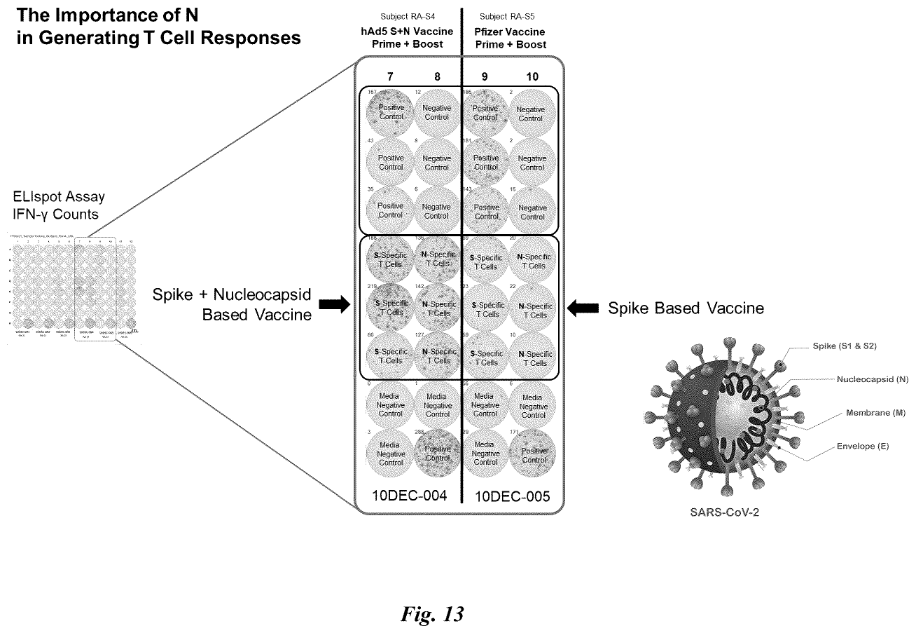

FIG. 12 validates the need for both S+N to be present to induce long-term memory B and T cells for a universal 2nd generation vaccine. SARS-CoV-2 infected patients are protected by cross reactive T cells without antibodies. hAd5 S+N vaccination induces memory B cells with complete protection following viral challenge in NHP. hAd5 S+N vaccination induces both T cell and cross-reactive memory B cells in healthy subjects. The importance of N in generating T cell responses is further disclosed in FIG. 13. As can be seen from this figure, the hAd5 S+N Vaccine Prime+Boost schedule as disclosed herein provides better and longer protection as compared to Spike based vaccine. Consequently, the inventors have surprisingly found that the vaccine compositions presented herein targeting both S and N of SARS-CoV2 exhibited unexpected cross-reactivity against a variety of other coronaviruses, and particularly against SARS-CoV1, MERS-CoV, OC43-CoV, and HKU1-CoV in addition to SARS-CoV2.

Embodiments of the present disclosure are further described in the following examples. The examples are merely illustrative and do not in any way limit the scope of the invention as claimed.

### EXAMPLE 1

With respect to the experiments performed and data presented, the following reagents and methods were employed in addition to well-known protocols:

Peptide pools (Pepmix™): 15-mer peptides that overlapped by 11 amino acids and spanned the entire protein sequence of the spike of SARS-CoV-2 (Wuhan, Alpha, Epsilon, Gamma and Beta) were purchased from JPT (JPT Peptide Technologies GmbH, Berlin, Germany).

ELISpot assay: ELISpot plates were coated with human IFNγ and IL-4 antibody (ImmunoSpot, Cleveland, USA) overnight at 4° C. Then, 300,000 PBMCs were seeded per well and stimulated for 44-48 h with SARS-CoV-2 Pepmix™ (2.5 μg/ml/peptide, JPT, Germany), Subsequently, the plates were developed according to kit's instructions (hIFNgIL4-2M/2, Immunospot). Plate were scanned and Spot forming units (SFU) were quantified using ImmunoSpot S6 Universal-V Analyzer with ImmunoSpot MultiSet AutoCount™ software.

### EXAMPLE 2: CYTOMETRIC BEAD ARRAY GENERATION

Conjugation of beads with Streptavidin: The Cytometric Bead Array (CBA) used in this analysis was constructed using spherotech 4 um and 5 um carboxy bluepak array kits (cat PAK-4067-8K and PAK-5067-10K respectively). The beads were functionalized by first conjugating Streptavidin (SA) to the beads via commonly employed 1-Ethyl-3-(3-dimethylaminopropyl)carbodiimide (EDC) chemistry. SA (southern biotech cat 7105-01) was buffered exchanged using pd-10 columns (Cytiva 17-0851-01) into PBS and diluted to 2 mg/mL. For conjugation, 10e8 spherotech particles were isolated by centrifugation at 10,000×g for 3 min. After carefully removing the supernatant, the bead pellet was resuspended in 0.5 mLs of SA in PBS. Following complete resuspension by pipetting, 0.5 mLs of 6 mM EDC dissolved in 0.05M MES buffer PH 5.0 was added, and the reaction mixture was rotated at room temperature overnight. After the conjugation reaction was complete, 0.1 mL of 1M tris PH 8.0 was added to quench the reaction. Following a 1 hr incubation rotating at RT, the beads were harvested by centrifugation as described above and washed twice in 1 mL of PBS. Following the final wash, beads were resuspended in 1 mL of PBS with 0.25% NaN3 and stored at 4° C. until use.

SA loading quality assurance: Following SA conjugation, quality control experiments were performed to determine the degree and uniformity (when multiple particle sizes and/or peak identities are used) of labeling by staining the SA-conjugated particles with fluorescently-labeled-biotinylated hemagglutinin (PR8). Individual array constituents were mixed and diluted to 1e6 of each particle/mL. 40 ml of serial dilutions of PR8 were prepared in a 96 well U bottom plates (costar 3797) ranging from 1 ug/mL to 2 ng/mL. 5 ml of the bead suspension was added, mixed by pipetting, and incubated for 15 min at RT. 200 ml of PBS was then added and the plate was centrifuged at 3000×g for 5 min. The beads were resuspended in 80 ml of PBS. Samples were then analyzed by flow cytometry.

Recombinant antigen absorption: Following the SA coupling and quality control procedures described above, biotinylated recombinant array antigens were passively absorbed onto the individual particles. For antigens used in this array configuration, a single biotin site was added enzymatically onto a carboxy terminal AVI tag. SA conjugated particles were harvested by centrifugation as described above and resuspended in 1 mg/mL of the biotinylated recombinant proteins in 1% BSA in PBS. Antigen loading was carried out by rotating overnight at 4° C. Following absorption, the beads were harvested by centrifugation as described, and washed twice with 1% BSA in PBS. Finally, the antigen coated beads were resuspended at 1e8 particles/mL 1% BSA in PBS, 0.25% NaN3 and stored at 4° C. until use.

Ig Standards: To construct indirect standard beads, bead peaks selected for each isotype were combined and biotinylated goat-anti Isotype F(ab)2 Abs (Southern Biotech: anti-IgM 2022-01, anti-IgA 2052-01, and anti-IgG 2042-01) were added at a concentration of 1 mg/mL. Standard bead preparations were washed, harvested, and stored as described for antigen coated beads.

### EXAMPLE 3: RECOMBINANT ANTIGEN PRODUCTION

Recombinant antigens used in CBA: Recombinant antigens used in this array include influenza H1 Ca09 hemagglutinin (HA) and b-coronavirus (CoV) Spike (SP), Spike subdomains (receptor binding domain (RBD) and N terminal domain (NTD)), and Nucleocapsid protein (N). The recombinant CoV S and N proteins were produced from sequences derived from the 5 known human infectious b-coronaviruses. These include the Wuhan/Washington strain of SARS-CoV-2 (abbreviated C), SARS1 (abbreviated S), MERS (abbreviated M), OC43 (abbreviated O), and HKU1 (abbreviated H). RBD and NTD SP subdomains were produced from sequences derived from Wuhan/Washington strain of SARS-CoV-2. It is contemplated that Influenza Hemagglutinin protein has at least about 80%, at least 85%, at least 90%, at least 95%, at least 99%, or 100% sequence identity to the polypeptide of SEQ ID NO:26.

Production of pre-fusion recombinant Spike (SP) protein: Ectodomain SP pre-fusion trimers (SARS-CoV-2 S₁₄₋₁₂₁₁) were produced by co-transfecting SP-AviTag and SP-6X-HisTag constructs into FreeStyle 293-F Cells at a 1:2 ratio. Transfected cells were cultured in FreeStyle 293 Medium for 3 days and recombinant SP trimers were purified from culture supernatant by FPLC using Nickel-affinity chromatography. Purified proteins were biotinylated in vitro using BirA enzyme.

In terms of the CoV Spike (SP) ectodomains, it is contemplated that SARS1-CoV Spike ectodomain (S SP) with AVI tag has at least about 80%, at least 85%, at least 90%, at least 95%, at least 99%, or 100% sequence identity to the polypeptide of SEQ ID NO:8. The S SP 6His protein is contemplated to have at least about 80%, at least 85%, at least 90%, at least 95%, at least 99%, or 100% sequence identity to the polypeptide of SEQ ID NO:9. The SARS-CoV2 Spike ectodomain (C SP) with AVI tag is contemplated to have at least about 80%, at least 85%, at least 90%, at least 95%, at least 99%, or 100% sequence identity to the polypeptide of SEQ ID NO:10. The C SP 6 His tag protein is contemplated to have at least about 80%, at least 85%, at least 90%, at least 95%, at least 99%, or 100% sequence identity to the polypeptide of SEQ ID NO:11. The MERS Spike ectodomain (M SP) 6His tag protein is contemplated to have at least about 80%, at least 85%, at least 90%, at least 95%, at least 99%, or 100% sequence identity to the polypeptide of SEQ ID NO:12. The M SP AVI Tag protein is contemplated to have at least about 80%, at least 85%, at least 90%, at least 95%, at least 99%, or 100% sequence identity to the polypeptide of SEQ ID NO:13. The OC43 Spike ectodomain (O SP) 6His tag protein is contemplated to have at least about 80%, at least 85%, at least 90%, at least 95%, at least 99%, or 100% sequence identity to the polypeptide of SEQ ID NO:14. The OC43 Spike ectodomain (O SP) 6His tag protein is contemplated to have at least about 80%, at least 85%, at least 90%, at least 95%, at least 99%, or 100% sequence identity to the polypeptide of SEQ ID NO:15. The HKU1 Spike ectodomain (H SP) 6His tag protein is contemplated to have at least about 80%, at least 85%, at least 90%, at least 95%, at least 99%, or 100% sequence identity to the polypeptide of SEQ ID NO:16. The H SP Avi Tag protein is contemplated to have at least about 80%, at least 85%, at least 90%, at least 95%, at least 99%, or 100% sequence identity to the polypeptide of SEQ ID NO:17.

Production of S subdomains: NTD (SARS-CoV-2 S₁₄₋₃₀₅) and RBD (SARS-CoV-2 S₃₁₉₋₅₄₁) monomers with a C-terminal dual AviTag/6X-HisTag sequence were produced by transfecting single constructs into FreeStyle 293-F Cells. Following a 3-day expression, subdomains were purified from culture supernatant by FPLC using nickel-affinity chromatography and biotinylated in vitro by addition of BirA.

In terms of the Spike subdomains, it is contemplated that Sars-CoV-2 receptor binding domain (C RBD) 6HIS with AVI tag has at least about 80%, at least 85%, at least 90%, at least 95%, at least 99%, or 100% sequence identity to the polypeptide of SEQ ID NO:18. The Sars-CoV-2 N-terminal domain (C NTD) is contemplated to have at least about 80%, at least 85%, at least 90%, at least 95%, at least 99%, or 100% sequence identity to the polypeptide of SEQ ID NO:19. The SARS1-CoV Receptor binding domain (S RBD) 6HIS AVI tag protein is contemplated to have at least about 80%, at least 85%, at least 90%, at least 95%, at least 99%, or 100% sequence identity to the polypeptide of SEQ ID NO:20.

Production of CoV nucleocapsid protein (N): Recombinant N containing the full-length N and tandem AviTag/6X-HisTag sequence were produced by co-transforming Rosetta cells with the N expression plasmid and an inducible BirA expression plasmid. Cells were grown in the presence of chloramphenicol, ampicillin, and streptomycin, induced with IPTG and supplemented with biotin. Biotinylated N protein was purified by FPLC using a nickel-affinity column and subsequent size exclusion chromatography.

For the N proteins, it is contemplated that the SARS-CoV Nucleocapsid protein (S NP) 6HIS AVI tag has at least about 80%, at least 85%, at least 90%, at least 95%, at least 99%, or 100% sequence identity to the polypeptide of SEQ ID NO:21. The SARS-CoV-2 Nucleocapsid protein (C NP) 6HIS AVI tag is contemplated to have at least about 80%, at least 85%, at least 90%, at least 95%, at least 99%, or 100% sequence identity to the polypeptide of SEQ ID NO:22. The MERS Nucleocapsid protein (M NP) 6HIS AVI tag is contemplated to have at least about 80%, at least 85%, at least 90%, at least 95%, at least 99%, or 100% sequence identity to the polypeptide of SEQ ID NO:23. The OC43 Nucleocapsid protein (O NP) 6HIS AVI tag is contemplated to have at least about 80%, at least 85%, at least 90%, at least 95%, at least 99%, or 100% sequence identity to the polypeptide of SEQ ID NO:24. The HKU1 Nucleocapsid protein (H NP) 6HIS AVI tag is contemplated to have at least about 80%, at least 85%, at least 90%, at least 95%, at least 99%, or 100% sequence identity to the polypeptide of SEQ ID NO:25.

Antibodies and standards: Detection of IG fluorescent goat polyclonal anti-IG F (ab′)2 secondaries, SouthernBiotech (IgM cat 2022-02, IgG cat #2062-09, IgA cat #2052-09) Isotype standards were generated by performing the array on mixtures of IG capture beads with 0.75× serial dilutions of purified human antibodies southern biotech (IgG cat #0150-01, IgM Cat #0158L-01, IgA cat #0155L-01) in ranging from 1 ug/mL to 1.3 ng/mL.

### EXAMPLE 4: CBA ASSAY

Serum samples were diluted into PBS ( 1/7150 for IgG detection, or 1/500 for IgM and IgA detection) and arrayed in 96 well u-bottom plates. A 5 μl suspension containing 5×1e5 of each antigen coated microparticles was added to the samples. In the case of Ig standards, anti-IgM, anti-IgA, and anti-IgG beads were added to 50 ml of the serial dilutions of standard Abs. The suspensions were mixed by pipetting and incubated for 15 min at room temperature. The beads were washed by the addition of 200 μl of PBS and centrifugation at 3000 g for 5 min at room temperature. The CBA particles were resuspended in a secondary staining solution consisting of the appropriate secondary diluted 1/400 in 1% BSA in PBS. The suspension was incubated for 15 min in the dark at room temperature. The beads were washed by the addition of 200 μl of PBS and pelleted by centrifugation at 3000 g for 5 min at room temperature. The particles were resuspended in 80 μl PBS and directly analyzed on a BD Cytoflex flow cytometer in plate mode at sample rate of 100 ml per minute. Sample collection was stopped following the acquisition of 75 μL. Following acquisition, the resulting FCS files were processed using the software described below.

### EXAMPLE 5: SAMPLE ANALYSIS

FCS processing: FCS files derived from the samples were analyzed using a custom software to automatically process FCS files to rapidly quantify the antibody reactivities of serum samples. This software was developed in Matlab (The Mathworks, Inc. Natick MA, USA) version R2020a on MacOS. It requires the Statistics and Machine Learning Toolbox, the Curve Fitting Toolbox and the Signal Processing Toolbox, and additional code from Matlab Central (www.mathworks.com/matlabcentral/).

Concentration determinations: The MFI data are extracted from an FCS file and transformed using the hyperbolic arcsine. Next, a forward-scatter vs. side-scatter plot is used to differentiate the different sized beads and intensity in the APC-cy7 channels as densities of points. These are automatically detected and events within these gates are annotated as distinct populations of beads. Finally, events from each bead gate are evaluated on the secondary isotype flow channel(s) r each bead feature and isotype.

Standard samples for each isotype and bead size are processed similarly and the resulting data are used to compute a four-parameter logistic (4PL) fit for each bead size/isotype/dilution. Finally, the 4PL fits are used to back-calculate concentration units for the MFI data, across the entire data set as a single, tabular text file containing the calculated Ig concentration data for all features in the array.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A kit for use in eliciting in a subject an immune response against a SARS-CoV-2 variant, the kit comprising: a recombinant vaccine composition having a first portion encoding a severe acute respiratory syndrome (SARS) coronavirus nucleocapsid protein (N) that is fused to an endosomal targeting sequence (N-ETSD), wherein the first portion comprises a nucleic acid encoding an amino acid sequence having 90% identity to SEQ ID NO:1 or SEQ ID NO:7, and a second portion encoding a SARS virus spike protein (S); and directions for use thereof.

2. The kit of claim 1, wherein the immune response comprises the generation of antibodies that bind to the Delta, Wuhan, Alpha, Epsilon, Gamma, and Beta variants of SARS-CoV2.

3. The kit of claim 1, wherein the immune response is generation of cytotoxic T cells that have cytotoxicity against different cells harboring Delta, Omicron, Wuhan, Alpha, Epsilon, Gamma, or Beta variants of SARS-CoV2.

4. The kit of claim 1, wherein the immune response is generation of memory T cells and/or memory B cells.

5. The kit of claim 1, wherein the endosomal targeting sequence of the N-ETSD is encoded at a 5′-end of the first portion and/or wherein the endosomal targeting sequence of the N-ETSD is encoded at a 3′-end of the first portion.

6. The kit of claim 1, wherein the S protein has an amino acid sequence having 90% identity to SEQ ID NO:3 or SEQ ID NO:4.

7. The kit of claim 1, wherein the second portion comprises a nucleotide sequence having 90% identity to SEQ ID NO:5 or SEQ ID NO:6.

8. The kit of claim 1, wherein the recombinant vaccine composition is formulated as a recombinant virus.

9. The kit of claim 8, wherein the recombinant virus is an adenovirus comprising an E1 gene region deletion and an E2b gene region deletion.

10. The kit of claim 1, wherein the recombinant vaccine composition is formulated as a recombinant RNA.

11. The kit of claim 1, wherein the recombinant vaccine composition is formulated as a recombinant DNA.

12. The kit of claim 1, further comprising a subunit vaccine comprising a recombinant protein of a coronavirus.

13. The kit of claim 1, further comprising a heat-inactivated coronavirus vaccine composition.

14. A kit for use in eliciting in a subject an immune response against a SARS-CoV-2 variant, the kit comprising: 1) a recombinant adenoviral vaccine composition comprising an E1 gene region deletion, an E2b gene region deletion, and a nucleic acid comprising a first portion encoding a severe acute respiratory syndrome (SARS) coronavirus nucleocapsid protein (N) that is fused to an endosomal targeting sequence (N-ETSD), wherein the first portion encodes an antigenic peptide having the sequence of SEQ ID NO:1 or SEQ ID NO:7, and a second portion encoding a SARS virus spike protein (S); 2) an self-amplifying self-adjuvant RNA vaccine comprising a ribonucleic acid encoding a severe acute respiratory syndrome (SARS) coronavirus spike protein (S), and 3) directions for use thereof.

15. The kit of claim 14, wherein the immune response comprises the generation of antibodies that bind to the Delta, Wuhan, Alpha, Epsilon, Gamma, and Beta variants of SARS-CoV2.

16. The kit of claim 14, wherein the immune response is generation of cytotoxic T cells that have cytotoxicity against different cells harboring Delta, Omicron, Wuhan, Alpha, Epsilon, Gamma, or Beta variants of SARS-CoV2.

17. A kit for use in eliciting in a subject an immune response against a SARS-CoV-2 variant, the kit comprising: 1) a recombinant adenoviral vaccine composition comprising an E1 gene region deletion, an E2b gene region deletion, and a nucleic acid comprising a first portion encoding a severe acute respiratory syndrome (SARS) coronavirus nucleocapsid protein (N) that is fused to an endosomal targeting sequence (N-ETSD), wherein the first portion encodes an antigenic peptide having the sequence of SEQ ID NO:1 or SEQ ID NO:7, and a second portion encoding a SARS virus spike protein (S); 2) a subunit vaccine comprising a recombinant protein of a corona virus; and 3) directions for use thereof.

18. The kit of claim 17, wherein the immune response comprises the generation of antibodies that bind to the Delta, Wuhan, Alpha, Epsilon, Gamma, and Beta variants of SARS-CoV2.

19. The kit of claim 17, wherein the immune response is generation of cytotoxic T cells that have cytotoxicity against different cells harboring Delta, Omicron, Wuhan, Alpha, Epsilon, Gamma, or Beta variants of SARS-CoV2.


SEQUENCE LISTING (26 sequences)

SEQ ID NO: 1 - protein, 516 aa, synthetic construct
  REGION 1..516: note = SARS-CoV2 Nucleocapsid protein tagged with ETSD signal
MLLLPFQLLAVLFPGGNSEDYKDDDDKGGGSGGGSGGGSGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGPGPGNLVPMVATVGPGPGMLIPIAVGGALAGLVLIVLIAYLIGKKHCSYQDIL

SEQ ID NO: 2 - DNA, 1551 bp, synthetic construct
  misc_feature 1..1551: note = SARS-CoV2 nucleocapsid tagged with ETSD
  source 1..1551: mol_type = other DNA
atgctgctgctgcccttccagttgctggctgtcctctttcccggcggcaactccgaggattacaaggacgacgacgacaagggtggaggctctggaggtggctctggtggaggttccggtggcggatctatgagcgacaacggtccccagaatcaaagaaatgcgcccagaattacattcggcggcccttctgatagcactggctcaaatcaaaacggggagagaagcggagccaggtccaaacagcggagaccccaaggcctgcctaataacaccgcttcctggttcacagctctgacgcaacacggcaaggaggatctgaagtttccacggggtcagggcgtcccgattaacacgaactctagcccagatgaccaaatagggtactacagaagagcgacaaggcggatcagaggaggcgatggaaaaatgaaggatctgtcccctaggtggtatttctattacctgggcacaggccctgaagctgggttgccttacggcgcaaacaaagatggaattatatgggtggccaccgagggggcgttgaacaccccaaaggatcacatcggaacgaggaatcccgccaacaatgctgctatagtgctccaactgccacagggaacaaccctgcctaagggcttctacgccgaggggagccgcggtggcagccaggccagctccagaagttcctcccgcagccggaacagctctagaaacagcactcccggcagctccagagggacaagcccagccagaatggccggcaatggcggcgacgctgccctcgcacttctgttgcttgatcggctcaatcaactcgaaagcaaaatgtccggcaagggacaacaacagcaaggacagaccgttacaaaaaaaagcgccgccgaggctagcaagaagcccagacagaagcgaaccgcaacaaaggcctataatgtaacacaagcctttggaaggcggggacccgaacagacccagggaaattttggcgaccaggaactgatccggcaagggacagactataaacattggccacagatagcgcaatttgctccctccgcctccgccttctttggcatgtcaagaataggcatggaagtaactccttctggaacctggctgacgtacactggggcaatcaagttggatgataaggaccctaatttcaaggaccaagttattttgctcaacaagcatatagacgcctacaagactttcccgcctaccgaacctaaaaaggataagaagaagaaagcagacgagacccaggccctgcctcaacggcaaaagaagcagcaaactgtgacactcctgcccgccgctgacttggatgatttttcaaaacagctccaacagagtatgagcagcgccgatagcacccaagctggaccgggtccgggcaacctggtgccgatggtggcgaccgtgggtccaggaccgggtatgctgatccccatcgccgtgggcggggccctggccggcctcgtgctgatcgtccttatcgcctacctcatcggcaagaagcactgctcatatcaggacatcctgtga

SEQ ID NO: 3 - protein, 1282 aa, synthetic construct
  REGION 1..1282: note = SARS-CoV2 spike protein with HA tag
MFVFLVLLPLVSSYPYDVPDYAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1298 aa, synthetic construct
  REGION 1..1298: note = SARS-CoV2 spike protein optimized for surface expression ("Sfusion")
MFVFLVLLPLVSSYPYDVPDYAGGGSGGGSGGGSGGGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3849 bp, synthetic construct
  misc_feature 1..3849: note = SARS-CoV2 spike protein with HA tag
  source 1..3849: mol_type = other DNA
atgttcgtttttctcgttctcctcccgcttgtgagcagctatccgtatgatgtgccggattatgcgcaatgtgtcaacctcaccacaaggacacagctccctcccgcatatacgaatagctttaccagaggcgtatactatcctgataaggtctttaggagctcagtactgcatagcactcaggatctcttcctgccgttcttcagtaatgttacttggtttcacgccattcatgtttccgggaccaatggcaccaaacggttcgataatccagtgcttcccttcaacgatggggtgtactttgccagcactgaaaaatctaatataattcggggatggattttcggaaccacactcgattccaagactcagtccctcttgatcgttaacaacgctactaatgttgtcattaaggtgtgtgagtttcagttctgcaacgaccctttcctgggtgtctactaccataaaaataacaagagctggatggagtccgaatttcgcgtctactcaagcgccaataattgcacttttgagtatgtgtcccagccctttttgatggatctggagggaaagcagggcaatttcaaaaatctgagagaattcgtttttaagaatatagatggatacttcaaaatctacagcaaacacacacccataaatcttgtgcgcgatcttccccagggcttcagcgcgttggaaccccttgttgacttgcccataggcatcaacattaccaggttccaaacgctgctcgccctccaccgcagctacttgacacccggggattccagctccggatggaccgccggcgccgcagcgtattatgtggggtacctgcaacccaggacatttttgctcaagtacaatgagaatgggaccatcacagatgcggtagactgtgcactggatccactcagcgaaactaaatgtaccctgaaaagctttaccgtggagaaaggaatctaccaaaccagcaacttcagggtccagcccactgaatccatcgttagatttccaaatataactaatttgtgtccatttggagaggtgttcaatgctacaaggttcgcgtctgtatacgcttggaaccggaagcgcatctcaaattgcgtggctgattatagcgttctttacaacagcgcttccttttccacgttcaagtgctatggtgtatccccgacaaagctgaatgacttgtgcttcaccaatgtgtatgcggattctttcgttattcgaggcgatgaagtcagacaaattgcgcctggccagaccggaaagattgccgactacaactataaactgccggacgactttactggttgcgtgatcgcttggaacagcaataatcttgatagtaaagttggaggaaactacaattacctctatagactgttcagaaagagcaacttgaagccattcgaacgggatatctctacggagatctatcaagctggcagcaccccctgcaatggtgtggaaggctttaattgttattttcctttgcagagctatggcttccaacctaccaacggagtgggctaccagccctacagagtggtggtgctcagctttgaactgctgcatgccccggccacagtttgcgggcccaaaaaaagcacgaatctggttaagaacaaatgcgtcaacttcaattttaatgggttgacaggtacaggcgtactgaccgaatccaacaaaaagttcctgccttttcagcagttcgggagagatatcgccgacactacagacgccgtcagggatccccaaacactcgaaattctggacatcacaccttgttccttcggcggggtatctgtgattactccgggcacaaataccagtaaccaggtagcggtgctttaccaggatgtcaactgtacggaagtacctgtcgctattcatgcggatcaactcactcctacctggagagtttattccactgggtccaacgtgtttcagacccgagccggctgcttgattggcgcggaacatgttaacaactcctacgaatgtgacatccctatcggagctggcatctgtgcttcctatcaaacgcaaacgaacagcccacggcgggccagatccgtagcctctcaaagcatcatcgcttatactatgtccttgggggctgaaaacagcgttgcctattccaacaatagcatcgctatccctaccaactttaccatttccgtgaccacagaaatactgccggtgagcatgacaaagacttctgtggactgtaccatgtatatatgcggcgatagcacagagtgttctaatttgctgctgcagtacggcagcttttgtacccaactcaacagagcacttacagggattgccgtcgagcaggataaaaacacccaggaggttttcgcccaggttaagcagatctacaagaccccaccaatcaaggatttcggcggcttcaatttttcccagatactgcccgatccttccaagccatccaaaaggagctttatagaggatctgctgttcaacaaggtgactctggccgacgctggctttatcaagcaatatggcgattgcctgggggatattgccgctagggaccttatctgcgctcaaaaattcaacggtcttaccgttctcccgcccctgctcaccgacgagatgatagcccagtacacgagcgcacttttggccggcacgataaccagcggctggacattcggtgccggggccgctcttcaaatcccctttgccatgcagatggcctacagatttaatgggataggcgtgacacaaaatgtcttgtatgaaaatcagaaactgattgcaaaccagtttaatagcgctattggcaagatccaagatagcctttcctccaccgcatccgctctgggaaagttgcaagacgtcgtgaatcaaaacgcccaagctctgaataccctcgtgaagcagcttagctccaactttggcgcgatatcctccgtgctgaacgatatcctgtccagattggacaaggtcgaggcagaagtccagatcgatagattgataaccggcagactccagtctctgcagacatatgtgactcagcagttgataagagcggccgaaatacgagcgtctgcaaatctcgcagcaacgaaaatgtcagagtgtgtattggggcaaagtaaaagagtagatttctgtggaaagggttaccatctgatgtcattcccccagtctgcaccacatggagtagtttttttgcatgtgacttatgtgcctgcccaggagaaaaatttcaccactgcacctgcgatctgtcatgacggcaaggcacatttccctagagaaggcgtcttcgtatcaaatggaacacactggtttgtaacccaaaggaacttttacgagccccaaattataactaccgacaacaccttcgtaagcggaaactgcgacgtcgttatagggatagtcaataatacggtctatgaccctcttcagccggaactggactcctttaaagaagaactggataagtacttcaagaaccatacgtctccggatgtggatctcggagatataagtggaatcaacgcaagcgtagtaaacattcagaaggagatagaccgactcaatgaggttgctaaaaacctgaacgaaagcttgatagacttgcaggagctgggtaagtacgaacagtacattaagtggccatggtatatctggttgggcttcatagcaggactcatagctatcgtcatggtgacaataatgctttgttgtatgaccagctgttgttcttgtctgaaaggctgctgcagctgtggcagctgttgtaaatttgacgaagatgattccgagcctgtgcttaagggcgtaaaactccactatacatga

SEQ ID NO: 6 - DNA, 3897 bp, synthetic construct
  misc_feature 1..3897: note = SARS-CoV2 spike protein optimized for surface expression ("Sfusion")
  source 1..3897: mol_type = other DNA
atgttcgtttttctcgttctcctcccgcttgtgagcagctatccgtatgatgtgccggattatgcgggtggaggctctggaggtggctctggtggaggttccggtggcggatctcaatgtgtcaacctcaccacaaggacacagctccctcccgcatatacgaatagctttaccagaggcgtatactatcctgataaggtctttaggagctcagtactgcatagcactcaggatctcttcctgccgttcttcagtaatgttacttggtttcacgccattcatgtttccgggaccaatggcaccaaacggttcgataatccagtgcttcccttcaacgatggggtgtactttgccagcactgaaaaatctaatataattcggggatggattttcggaaccacactcgattccaagactcagtccctcttgatcgttaacaacgctactaatgttgtcattaaggtgtgtgagtttcagttctgcaacgaccctttcctgggtgtctactaccataaaaataacaagagctggatggagtccgaatttcgcgtctactcaagcgccaataattgcacttttgagtatgtgtcccagccctttttgatggatctggagggaaagcagggcaatttcaaaaatctgagagaattcgtttttaagaatatagatggatacttcaaaatctacagcaaacacacacccataaatcttgtgcgcgatcttccccagggcttcagcgcgttggaaccccttgttgacttgcccataggcatcaacattaccaggttccaaacgctgctcgccctccaccgcagctacttgacacccggggattccagctccggatggaccgccggcgccgcagcgtattatgtggggtacctgcaacccaggacatttttgctcaagtacaatgagaatgggaccatcacagatgcggtagactgtgcactggatccactcagcgaaactaaatgtaccctgaaaagctttaccgtggagaaaggaatctaccaaaccagcaacttcagggtccagcccactgaatccatcgttagatttccaaatataactaatttgtgtccatttggagaggtgttcaatgctacaaggttcgcgtctgtatacgcttggaaccggaagcgcatctcaaattgcgtggctgattatagcgttctttacaacagcgcttccttttccacgttcaagtgctatggtgtatccccgacaaagctgaatgacttgtgcttcaccaatgtgtatgcggattctttcgttattcgaggcgatgaagtcagacaaattgcgcctggccagaccggaaagattgccgactacaactataaactgccggacgactttactggttgcgtgatcgcttggaacagcaataatcttgatagtaaagttggaggaaactacaattacctctatagactgttcagaaagagcaacttgaagccattcgaacgggatatctctacggagatctatcaagctggcagcaccccctgcaatggtgtggaaggctttaattgttattttcctttgcagagctatggcttccaacctaccaacggagtgggctaccagccctacagagtggtggtgctcagctttgaactgctgcatgccccggccacagtttgcgggcccaaaaaaagcacgaatctggttaagaacaaatgcgtcaacttcaattttaatgggttgacaggtacaggcgtactgaccgaatccaacaaaaagttcctgccttttcagcagttcgggagagatatcgccgacactacagacgccgtcagggatccccaaacactcgaaattctggacatcacaccttgttccttcggcggggtatctgtgattactccgggcacaaataccagtaaccaggtagcggtgctttaccaggatgtcaactgtacggaagtacctgtcgctattcatgcggatcaactcactcctacctggagagtttattccactgggtccaacgtgtttcagacccgagccggctgcttgattggcgcggaacatgttaacaactcctacgaatgtgacatccctatcggagctggcatctgtgcttcctatcaaacgcaaacgaacagcccacggcgggccagatccgtagcctctcaaagcatcatcgcttatactatgtccttgggggctgaaaacagcgttgcctattccaacaatagcatcgctatccctaccaactttaccatttccgtgaccacagaaatactgccggtgagcatgacaaagacttctgtggactgtaccatgtatatatgcggcgatagcacagagtgttctaatttgctgctgcagtacggcagcttttgtacccaactcaacagagcacttacagggattgccgtcgagcaggataaaaacacccaggaggttttcgcccaggttaagcagatctacaagaccccaccaatcaaggatttcggcggcttcaatttttcccagatactgcccgatccttccaagccatccaaaaggagctttatagaggatctgctgttcaacaaggtgactctggccgacgctggctttatcaagcaatatggcgattgcctgggggatattgccgctagggaccttatctgcgctcaaaaattcaacggtcttaccgttctcccgcccctgctcaccgacgagatgatagcccagtacacgagcgcacttttggccggcacgataaccagcggctggacattcggtgccggggccgctcttcaaatcccctttgccatgcagatggcctacagatttaatgggataggcgtgacacaaaatgtcttgtatgaaaatcagaaactgattgcaaaccagtttaatagcgctattggcaagatccaagatagcctttcctccaccgcatccgctctgggaaagttgcaagacgtcgtgaatcaaaacgcccaagctctgaataccctcgtgaagcagcttagctccaactttggcgcgatatcctccgtgctgaacgatatcctgtccagattggacaaggtcgaggcagaagtccagatcgatagattgataaccggcagactccagtctctgcagacatatgtgactcagcagttgataagagcggccgaaatacgagcgtctgcaaatctcgcagcaacgaaaatgtcagagtgtgtattggggcaaagtaaaagagtagatttctgtggaaagggttaccatctgatgtcattcccccagtctgcaccacatggagtagtttttttgcatgtgacttatgtgcctgcccaggagaaaaatttcaccactgcacctgcgatctgtcatgacggcaaggcacatttccctagagaaggcgtcttcgtatcaaatggaacacactggtttgtaacccaaaggaacttttacgagccccaaattataactaccgacaacaccttcgtaagcggaaactgcgacgtcgttatagggatagtcaataatacggtctatgaccctcttcagccggaactggactcctttaaagaagaactggataagtacttcaagaaccatacgtctccggatgtggatctcggagatataagtggaatcaacgcaagcgtagtaaacattcagaaggagatagaccgactcaatgaggttgctaaaaacctgaacgaaagcttgatagacttgcaggagctgggtaagtacgaacagtacattaagtggccatggtatatctggttgggcttcatagcaggactcatagctatcgtcatggtgacaataatgctttgttgtatgaccagctgttgttcttgtctgaaaggctgctgcagctgtggcagctgttgtaaatttgacgaagatgattccgagcctgtgcttaagggcgtaaaactccactatacatga

SEQ ID NO: 7 - protein, 473 aa, SARS-CoV2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGPGPGNLVPMVATVGPGPGMLIPIAVGGALAGLVLIVLIAYLIGKKHCSYQDIL

SEQ ID NO: 8 - protein, 1249 aa, synthetic construct
  REGION 1..1249: note = SARS1-CoV Spike ectodomain (S SP) AVI tag; Mutations: Furinecleavage, Diproline
MEFGLSWLFLVAILKGVQCEVSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKGGGSGYIPEAPRDGQAYVRKDGEWVLLSTFLGSGLNDIFEAQKIEWHE

SEQ ID NO: 9 - protein, 1240 aa, synthetic construct
  REGION 1..1240: note = S SP 6His tag; Mutations: Furine cleavage, Diproline
MEFGLSWLFLVAILKGVQCEVSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKGGGSGYIPEAPRDGQAYVRKDGEWVLLSTFLGSHHHHHH

SEQ ID NO: 10 - protein, 1267 aa, synthetic construct
  REGION 1..1267: note = SARS-CoV2 Spike ectodomain (C SP) AVI tag; Mutations: furinecleavage, Diproline
MEFGLSWLFLVAILKGVQCEVQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGGGSGYIPEAPRDGQAYVRKDGEWVLLSTFLGSGLNDIFEAQKIEWHE

SEQ ID NO: 11 - protein, 1258 aa, synthetic construct
  REGION 1..1258: note = C SP 6 His tag; Mutations: furine cleavage, Diproline
MEFGLSWLFLVAILKGVQCEVQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGGGSGYIPEAPRDGQAYVRKDGEWVLLSTFLGSHHHHHH

SEQ ID NO: 12 - protein, 1336 aa, synthetic construct
  REGION 1..1336: note = MERS Spike ectodomain (M SP) 6His tag; Mutations: Furinecleavage, Diproline
MEFGLSWLFLVAILKGVQCEVVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKGGGSGYIPEAPRDGQAYVRKDGEWVLLSTFLGSHHHHHH

SEQ ID NO: 13 - protein, 1345 aa, synthetic construct
  REGION 1..1345: note = M SP AVI Tag; Mutations: Furine cleavage, Diproline
MEFGLSWLFLVAILKGVQCEVVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKGGGSGYIPEAPRDGQAYVRKDGEWVLLSTFLGSGLNDIFEAQKIEWHE

SEQ ID NO: 14 - protein, 1310 aa, synthetic construct
  REGION 1..1310: note = OC43 Spike ectodomain (O SP) 6His tag; Mutations: Furinecleavage, Diproline
MEFGLSWLFLVAILKGVQCEVVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAVDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNGGSGGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQFSGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLLGGGSGYIPEAPRDGQAYVRKDGEWVLLSTFLGSHHHHHH

SEQ ID NO: 15 - protein, 1319 aa, synthetic construct
  REGION 1..1319: note = O SP AVI tag; Mutations: Furine cleavage, Diproline
MEFGLSWLFLVAILKGVQCEVVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAVDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNGGSGGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQFSGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLLGGGSGYIPEAPRDGQAYVRKDGEWVLLSTFLGSGLNDIFEAQKIEWHE

SEQ ID NO: 16 - protein, 1328 aa, synthetic construct
  REGION 1..1328: note = HKU1 Spike ectodomain (H SP) 6His tag; Mutations: Furinecleavage, Diproline
MEFGLSWLFLVAILKGVQCEVVIGDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSGGSGSSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDALEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNGGGSGYIPEAPRDGQAYVRKDGEWVLLSTFLGSHHHHHH

SEQ ID NO: 17 - protein, 1337 aa, synthetic construct
  REGION 1..1337: note = H SP Avi Tag; Mutations: Furine cleavage, Diproline
MEFGLSWLFLVAILKGVQCEVVIGDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSGGSGSSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDALEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNGGGSGYIPEAPRDGQAYVRKDGEWVLLSTFLGSGLNDIFEAQKIEWHE

SEQ ID NO: 18 - protein, 266 aa, synthetic construct
  REGION 1..266: note = Sars-CoV-2 receptor binding domain (C RBD) 6HIS AVI tag
MEFGLSWLFLVAILKGVQCEVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGLNDIFEAQKIEWHEHHHHHH

SEQ ID NO: 19 - protein, 335 aa, synthetic construct
  REGION 1..335: note = Sars-CoV-2 N-terminal domain (C NTD)
MEFGLSWLFLVAILKGVQCEVQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSGGLNDIFEAQKIEWHEHHHHHH

SEQ ID NO: 20 - protein, 265 aa, synthetic construct
  REGION 1..265: note = SARS1-CoV Receptor binding domain (S RBD) 6HIS AVI tag
MEFGLSWLFLVAILKGVQCEVRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFGGLNDIFEAQKIEWHEHHHHHH

SEQ ID NO: 21 - protein, 446 aa, synthetic construct
  REGION 1..446: note = SARS-CoV Nucleocapsid protein (S NP) 6HIS AVI tag
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQAGGLNDIFEAQKIEWHELEHHHHHH

SEQ ID NO: 22 - protein, 443 aa, synthetic construct
  REGION 1..443: note = SARS-CoV-2 Nucleocapsid protein (C NP) 6HIS AVI tag
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGLNDIFEAQKIEWHELEHHHHHH

SEQ ID NO: 23 - protein, 435 aa, synthetic construct
  REGION 1..435: note = MERS Nucleocapsid protein (M NP) 6HIS AVI tag
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTDGGLNDIFEAQKIEWHEHHHHHH

SEQ ID NO: 24 - protein, 470 aa, synthetic construct
  REGION 1..470: note = OC43 Nucleocapsid protein (O NP) 6HIS AVI tag
MSFTPGKQSSSRASSGNRSGNGILKWADQSDQFRNFQTRGRRAQPKQTATSQQPSGGNVVPHYSWFSGITQFQKGKEFEFAEGQGVPIAPGVPATEAKGYWYRHNRRSFKTADGNQRQLLPRWYFYYLGTGPHAKDQYGTDINGVYWVASNQADVNTPADIVDRDPSSDEAIPTRFPPGTVLPQGYYIEGSGRSAPNSRSTSRTSSRASSAGSRSRANSGNRTPTSGVTPDMADQIASLVLAKLGKDATKPQQVTKHTAKEVRQKILNKPRQKRSPNKQCTVQQCFGKRGPNQNFGGGEMLKLGTSDPQFPILAELAPTAGAFFFGSKLELAKVQNLSGNPDEPQKDVYELRYNGAIRFDSTLSGFETIMKVLSENLNAYQQQDGMMNMSPKPQRQRGHKNGQGENDNISVAVPKSRVQQNKSIELTAEDISLLKKMDEPFTEDTSEIGGLNDIFEAQKIEWHEHHHHHH

SEQ ID NO: 25 - protein, 463 aa, synthetic construct
  REGION 1..463: note = HKU1 Nucleocapsid protein (H NP) 6HIS AVI tag
MSYTPGHYAGSRSSSGNRSGILKKTSWADQSERNYQTFNRGRKTQPKFTVSTQPQGNTIPHYSWFSGITQFQKGRDFKFSDGQGVPIAFGVPPSEAKGYWYRHSRRSFKTADGQQKQLLPRWYFYYLGTGPYANASYGESLEGVFWVANHQADTSTPSDVSSRDPTTQEAIPTRFPPGTILPQGYYVEGSGRSASNSRPGSRSQSRGPNNRSLSRSNSNFRHSDSIVKPDMADEIANLVLAKLGKDSKPQQVTKQNAKEIRHKILTKPRQKRTPNKHCNVQQCFGKRGPSQNFGNAEMLKLGTNDPQFPILAELAPTPGAFFFGSKLDLVKRDSEADSPVKDVFELHYSGSIRFDSTLPGFETIMKVLEENLNAYVNSNQNTDSDSLSSKPQRKRGVKQLPEQFDSLNLSAGTQHISNDFTPEDHSLLATLDDPYVEDSVAGGLNDIFEAQKIEWHEHHHHHH

SEQ ID NO: 26 - protein, 569 aa, synthetic construct
  REGION 1..569: note = Influenza Hemagglutinin:California_VRDL7_2009 H1N1 HA (CA09) 6Histag
MGSLQPLATLYLLGMLVASCLGRLDTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPPTSADQQSLYQNADAYVFVGTSRYSKKFKPEIAIRPKVRGQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESKRMKQIEDKIEEIESKQKKIENEIARIKKGGGHHHHHH